(19) United States
(12) Patent Application Publication
(10) Pub. No.: US 2015/0051460 A1
(43) Pub. Date: Feb. 19, 2015

(54) SYSTEM AND METHOD FOR LOCATING BLOOD VESSELS AND ANALYSING BLOOD

(71) Applicants: NOOPUR SAXENA, BANGALORE (IN); SULAKSHNA SAXENA, BANGALORE (IN)

(72) Inventors: Sulakshna Saxena, Bangalore (IN); Noopur Saxena, Bangalore (IN)

(21) Appl. No.: 14/388,695

(22) PCT Filed: Apr. 4, 2013
(86) PCT No.: PCT/IN2013/000228

§ 371 Date: Sep. 26, 2014

(30) Foreign Application Priority Data
Apr. 4, 2012 (IN) 1363/CHE/2012

(51) Int. Cl. A61B 5/00; A61B 5/1455; A61B 5/06

(52) U.S. Cl. CPC A61B 5/489; A61B 5/061; A61B 5/1455; A61B 5/742
USPC 600/322; 600/407; 600/476; 600/424

(57) ABSTRACT

A non-invasive system (100) and method for locating blood vessel and analyzing blood of a subject under observation have been disclosed. The system (100) comprises a processor (108), an imaging module in communication with said processor (108) to capture at least a portion of a subject under observation and a display module (112) in communication with said processor to display said portion of the subject under observation (116). In further embodiments said processor (108) is configured to receive data from said imaging module and to construct a surface map of said portion of said section of said surface under observation (116).

### CROSS REFERENCE TO RELATED PATENT APPLICATIONS

The present patent application is a National Phase Application of PCT International Application No. PCT/IN2013/000228 having an International filing date of 4 Apr. 2013 with the title “System and Method for Locating Blood Vessels and Analysing Blood” and designating the United States of America, and deriving priority on an Indian Provisional Patent Application No. 1363/CHE/2012 filed on 4 Apr. 2012 with the title “System and Method for Obtaining and Studying Venous Map and Venous Blood Analysis”, and the contents of which are incorporated herein by reference in entirety.

### BACKGROUND

1. Technical Field

The embodiments herein generally relate to a medical device and more particularly but not exclusively to a non-invasive system and a method for locating blood vessel and analyzing blood.

2. Description of the Related Art

Venipuncture is an act of puncturing vein with a needle, usually for the purpose of adding medications to the blood or removing blood. Blood may be removed for the purpose of analyzing, donating, storing or therapeutically reducing the amount of blood in the body. Although, venipuncture is one of the most commonly performed process in medical industry, there are several potential complications related to venipuncture. Conventionally locating blood carrying veins in human body has been directed to physical and visual observation of the veins by experienced medical personnel for the insertion of blood drawing needles.

In conventional method, venipuncture is performed by manually identifying the blood carrying vein in human body and puncturing the vein by needle. Manual identification of vein may include the process of locating the vein by restricting the blood supply from the body-part. The insufficient blood supply from body-part results in the increase of blood accumulated in that area. Further, the increase of blood accumulated results in subject's veins becoming more visible. Furthermore, the whole process of restricting the blood supply to the body-part is performed by using a temporary tourniquet. Tourniquet is a compressing device that is configured to apply pressure circumferentially upon the skin and therefore also to underlying tissues of limb. However, the use of tourniquet results in extreme discomfort to the patient as it causes pain to the patient.

Further, the conventional method of identifying blood carrying vessels is difficult to perform on collapsed patient, trauma patient, obese patients, children especially with baby fat, elderly people, dehydrated patients, dark skin-tone people and the like. Furthermore, the accuracy of blood carrying vessels identified by the conventional method depends on the medical personnel's expertise. In most occasions, the carelessness/inexperience of medical personnel will result in insertion of needle in a wrong vein, missed puncture, improper puncture, and/or double puncture. The consequences of missed puncture include the need for repeated puncture thereby causing discomfort and pain to the patient. Also, when a bigger needle is used the puncturing may result in vessel bursting thereby rendering the site useless. Sometime, even with a proper needle the puncture may not happen at the center of the vein and the insertion may just touch the vein tangentially causing damage to the vein which is referred as improper puncturing. Further, a double puncture may be caused when the needle is inserted at a wrong angle, consequently leading to vein damage. The repeated puncture will result in loss of time in administering a life saving drug. Further, a missed puncture may result in a permanent nerve injury. Further multiple punctures to veins increase the risk of infection proportionately. Further, the conventional method is directed only to identify blood carrying veins and adding medications and drawing the blood. However, the analysis on the blood drawn is performed separately after drawing the blood and is time consuming. Further, the conventional method does not provide display or portray of venous map of the patient, whereas the venous map could be utilized with a pre-compiled catalogue of venous image maps by a medical personnel to examine the patients, for educational purposes and to provide a database of gathered information which could be used for further studies.

Therefore, there is a need for a non-invasive system and method for locating appropriate blood carrying veins. Further, there is a need to provide a system for locating veins which can obviate aforementioned drawbacks.

The above mentioned shortcomings, disadvantages and problems are addressed herein and which will be understood by reading and studying the following specification.

### OBJECTIVES OF THE EMBODIMENTS

The primary object of this invention is to provide a non-invasive system for locating blood vessels and analyzing blood.

Another object of the invention is to provide a system for non-invasively analyzing the blood and other fluids like enzymes, saliva and so on with relative ease.

Yet another object of the invention is to provide a cost effective system for locating appropriate blood carrying vessels and analyzing the blood and other fluids like enzymes, saliva and so on.

Yet another object of the invention is to provide a non-invasive system to characterize the vein in terms of width, depth, and straightness, and determine right needle size based on the aforementioned parameters and also the right elevation and azimuth angle for puncturing using this needle.

Yet another object of the invention is to provide a visual feedback of the blood vessel and the needle during an insertion/a procedure.

Yet another object of the invention is to provide a method for locating appropriate blood vessels and analyzing the blood and other fluids like enzymes, saliva, and so on.

These and other objects of the embodiments herein will be better appreciated and understood when considered in conjunction with the following description and the accompanying drawings.

### SUMMARY

The embodiments herein provide a non-invasive system for locating blood vessel and analyzing blood is disclosed. The system comprises a processor, an imaging module in communication with said processor to capture at least a portion of a subject under observation and a display module in communication with said processor to display said portion of the subject under observation. In further embodiments said processor is configured to receive data from said imaging module and to construct a surface map of said portion of said section of said surface under observation.

According to one embodiment herein, a method for locating blood vessel and analyzing blood is provided. The method includes providing a processor. Further, the method includes providing an imaging module in communication with said processor to capture at least a portion of a subject under observation. Furthermore the method includes providing a display module in communication with said processor to display said portion of the subject under observation.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 is a block diagram of non-invasive system for locating and highlighting the blood vessels of a subject under observation and analyzing the blood characteristics thereof, according to one embodiment herein.

FIG. 2 is a flow chart describing the steps involved in the method for locating and highlighting the blood vessels of a subject under observation and analyzing the blood characteristics thereof, according to one embodiment herein.

FIG. 3 depicts the steps involved in controlling the amount of light projected towards the subject under observation, according to one embodiment herein.

FIG. 4 is a flow chart illustrating the steps involved in tracking a needle piercing the subject under observation, according to one embodiment herein.

FIG. 5 is a flow chart illustrating the steps involved in statically determining an appropriate puncture spot on the surface of the subject of interest, according to one embodiment herein.

FIG. 6 is a flow chart illustrating the steps involved in dynamically determining an appropriate puncture spot on the surface of the subject of interest, according to one embodiment herein.

FIG. 7 is a flow chart illustrating the steps involved in analyzing the blood composition of the subject wider observation, according to one embodiment herein.

FIG. 8 is a flow chart illustrating the steps involved in automatic positioning of the imaging module for capturing an image having clarity, according to one embodiment herein.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

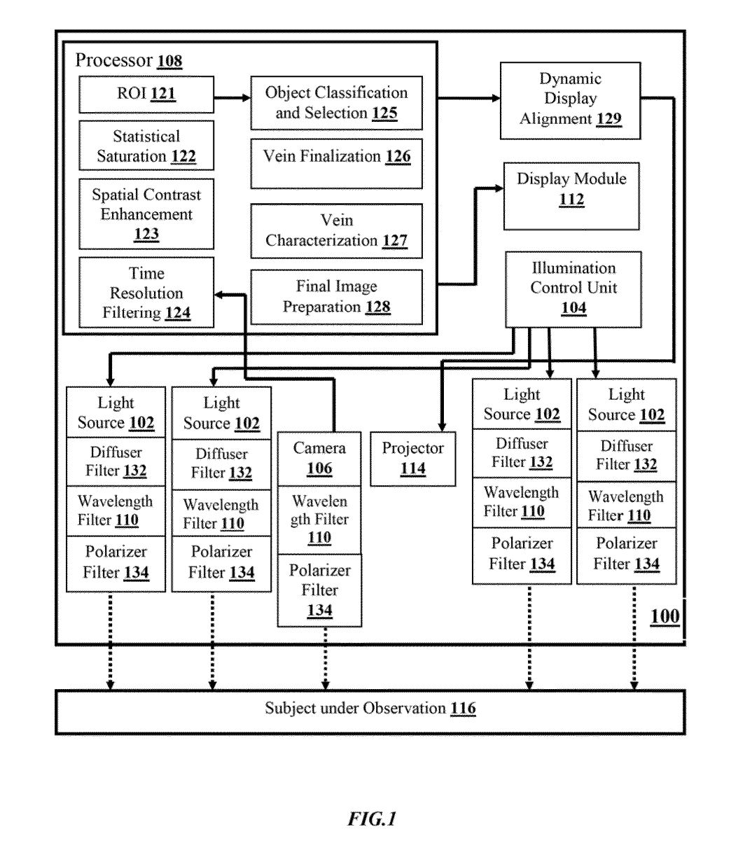

The embodiments herein achieve a non-invasive system (100) for locating blood vessel and analyzing blood. Referring now to the drawings, and more particularly to FIGS. 1 to 8, where similar reference characters denote corresponding features consistently throughout the figures, there are shown embodiments.

FIG. 1 depicts a block diagram of a non-invasive system (100) for locating, an appropriate blood vessel, according to one embodiment herein. The system (100) includes a processor (108), an imaging module and a display module (112). The imaging, module further includes light source (102) a control unit (104) a camera (106), a wavelength filter unit (110), a projector (114) and a cooling complex embedded inside control unit (not shown). The light source (102) is configured to emit a plurality of light signals towards a subject under observation (116). In one embodiment of the present disclosure, the subject under observation (116) is a part of human body where the blood vessel has to be identified. In another embodiment, the subject under observation (116) is an animal body where the blood vessels have to be located. In an embodiment, the light source (102) emits broad spectrum of light signal which includes but are not limited to visible light, Near Infrared (NIR), Infrared and other light wavelengths. In one embodiment the wavelength of the light source (102) varies between 700 nm to 1100 nm. In one embodiment the light source (102) is provided with at least one of specific wavelengths of 720 nm, 840 nm, 850 nm, 855 nm, 920 nm, 925 nm, 928 nm, 976 nm, 980 nm, 984 nm, 992nm, 1052 nm, 1050 nm and 1060 nm to generate specific illumination on the subject. In one embodiment each LED of the light source (102) could be of different wavelength. In another embodiment, the light source (102) includes source of light which includes but are not limited to Xenon bulb, Krypton bulb, Light Emitting Diode (LED), Halogen bulb, Laser light and so on. However, it is also within the scope of invention, that the light source (102) will include any other type of source that emits light of different wavelengths without otherwise deterring the intended function of the light source (102) as can be deduced from this description. Further, the light emitted by the light source (102) is directed towards the subject under observation (116) such that the directed light is reflected from the subject under observation (116). The control unit (104) is provided in communication with the light source (102) and configured to control at least one of intensity, pattern, curvature and wavelength of light emitted from the light, source (102). Further, at least one of intensity, pattern, curvature and wavelength of light emitted from the light source (102) is dynamically adjusted based on the skin tone, curvature and/or composition of the subject under observation, thereby providing better visualization of blood vessels. Further, the wavelength filter unit (110) along with a diffuser filter (132) and a polarizer filter (134) is provided in the path of directed light and reflected light. The wavelength filter unit (110) is configured to facilitate the passage of light with certain wavelength(s) that is useful for image processing. In an embodiment, the wavelength filter unit (110) is a band pass optical wavelength filter that is configured to allow light having preferred wavelength. In one embodiment a narrow band wavelength filtering technique is used for better visualization of the subject under observation. However, it is also within the scope of invention that the wavelength filter unit (110) may include any other type of wavelength filters as per the preferred wavelength of light. In one embodiment the system (100) consists of an independent, or a separate wavelength filter for each light path. Further, each wavelength filter may be provided with different characteristics to obtain desired light characteristics. Furthermore, an array of wavelength filters may be provided in the light path to obtain desired intensity/pattern or wavelength of light. Further, in another embodiment, the wavelength filter unit (110) is selected from a group that includes but not limited to long pass wavelength filter, short pass wavelength filter, narrow-band wavelength filter, and notch wavelength filter and the like.

According to one embodiment herein, the camera (106) is configured to receive the reflected light signal from the subject under observation (116). In an embodiment, the camera is selected from a group that includes but not limited to a standard complementary metal oxide semiconductor (CMOS) and Charged coupled device (CCD) cameras. However, it is also within the scope of invention that the camera (106) may be selected from any other type of camera without otherwise deterring the intended function of the system (100) as can be deduced from this description. Further, in another embodiment, especially for generating three-dimensional images plurality of cameras (106) is provided to receive the reflected light from the subject under observation (116).

According to one embodiment herein, the processor (108) is configured to facilitate functioning of all other components of the system (100). The processor (108) receives the information of the light reflected from the subject under observation (116) through camera (106). In one embodiment the processor (108) is configured with time, resolution filtering module (124), contrast enhancement module (123), hard contrast module (122), a region of interest (121), object classification and selection module (125), in finalization (126), vein characterization (147), final image preparation (128), and a dynamic, display alignment module (129). In an embodiment the aforementioned modules are displaced independently. Further, the processor (108) is configured to generate an image signal based in the light reflected from the subject under observation (116). In an embodiment, the processor (108) is programmed to generate image signal based on the light reflected from the subject under observation (116). Further, display (112) is provided in communication with the processor (108) and configured to display an image based on the image signal generated by the processor (108). The display (112) is selected from the display devices that include but are not limited to Liquid Crystal Display device, LED display device, OLED display device, TOLED display device and heads-up display. However, it is also within the scope of invention that the display (112) could be selected from any other type of display device without otherwise deterring the intended function of the display (112) as can be deduced from this description. In an embodiment, the projector (114) is provided in communication with processor (108), such that the projector (114) receives generated image from the processor (108) and projects it on to the display (112). In an embodiment, the image received by the projector (114) is dynamically aligned to ensure that the image is displayed at the right location. The dynamic alignment is performed by projecting a pre-determined fixed or varying pattern by projector (114) and reading it hack from the camera (106) and based on that determining the alignment parameters. In another embodiment, the projector (114) is configured to receive generated image from the processor (108) and project it back on to the subject under observation (116). However, it is also within the scope of invention that the projector (114) can be configured to project the generated image anywhere based on the requirement of the user of system (100). In another embodiment, the projector (114) is selected from a group that includes but not limited to DLP and Laser Projectors.

According to one embodiment herein, the processor (108) includes a memory, at least one input peripheral and at least one output peripheral. The input peripheral of processor (108) is provided in communication with the camera (106). Further, the output peripheral of processor (108) is provided in communication with light source (102), control unit (104), display (112) and projector (114). Further, the processor (108) is configured to receive the information on reflected light from subject under observation (116), from camera 102. Furthermore, the processor (108) is programmed to process the received information and generate image of the subject under observation (116) based on the reflected light. In an embodiment, the processor (108) controls the control unit (104) to adjust the characteristics of light to improve visibility of the image obtained. For example, varying at least one of intensity, pattern, curvature and wavelength of light from light source 102 might result in variation in the image contrast and the processor (108) is configured to vary at least one of intensity, pattern, curvature and wavelength of light using the control unit (104) based on the image contrast required. A better contrast enables a better processing of the obtained images. In another embodiment, the user can manually adjust at least one of intensity, pattern, curvature and wavelength of light based on the image contrast required. In another embodiment, if user uses the system (100) for venipuncture process, the light from the light source (102) is directed to a part of human body where the blood vessels are to he identified. Further, the image generated might include image of blood vessels which include arteries, veins and capillaries. Further the image may include skin, tissues and the like. In another embodiment, the processor (108) is configured to facilitate frame segmentation of the image generated. In another embodiment, the processor (108) is configured to identify the region of interest. In another embodiment, the processor (108) is configured to locate objects such as hands, needle and blood vessels and the like, to provide better visualization. In another embodiment, the processor (108) is configured to remove undesirable portions such as background of subject under observation area from the generated image. In yet another embodiment, the processor (108) is programmed to facilitate post processing in order to improve the image quality. The embodiment may include providing pseudo-tactical colorization to the final image for user convenience/better visibility. In another embodiment, the processor (108) is configured to enable dynamic alignment using (129) of the display image with respect to the acquired image.

According to one embodiment herein, the processor (108) is configured to detect the interested vessel or vein using an object classification and selection module (125). In one embodiment the colors of the vessel or vein are inverted (e.g. to green, blue) to provide a better visualization. This facilitates in providing a better visualization for thin veins in human body. In one embodiment the object classification and selection module (OCS) provides continuous feedback to a spatial contrast enhancement module (SCE) such that the SCE knows which part of the frame needs more/less enhancement. In another embodiment the processor (108) is configured with a SS module. Based on the feedback from the segmentation nodule, the hard contrast module provides a statistical saturation in the image generated. This statistical saturation increases he image contrast to a desired level. In one embodiment the hard contrast module takes input from the segmentation module to decide the level of statistical saturation to be provided for the image.

According to one embodiment herein, the processor includes a real time collaboration module. This module provides a real time streaming of a video to a third party present: elsewhere. Using this technique, for example, a nurse can consult a senior doctor in case she is not able to make the decision on inserting the needle to a subject. The final image can be transferred in two ways, one of being a single final image and the other being multi-stream image. In a multi-stream image transfer a base image is transferred separately and then each of the additional information is transferred separately in a different stream. At the receive end all the streams are combined based on the users preference to create a final image.

According to one embodiment herein, the processor (108) includes a time resolution-wavelength filtering module for SNR (Signal to Noise Ratio) improvement. There is always a micro shaking in the images that are captured in a normal setup. A shaking could occur due to shake in the camera-holder or due to shake in the subject of interest. This phenomenon occurs more in low-light scenario where the shutter of the camera has to be kept open for a longer time to compensate for the low light level. In one embodiment this issue is addressed by implementing a time resolution cleaning of the image. In an embodiment the images are captured at a faster rate (for example, 5× the processing frame rate). And these images are then analyzed to obtain a single sharper image.

According to one embodiment herein, the process (108) includes a Previous Frame Feedback Module (PFFM) which caches the knowledge from previous frames and applies it to enhance the contrast and detect the region of interest more efficiently in the current frame. In one embodiment it is assumed that subject and/or the device has not moved or changed drastically. In an embodiment if a significant change is detected in the input image, the Past Frame Feedback Module automatically shuts-off for the current frame and it resumes from next frame.

According to one embodiment herein, the system (100) is configured to display the depth and width of the vein of user's interest in viewing. In another embodiment a needle tracking and insertion detection module is provided in the system (100). This module is used in tracking the needle. In an embodiment the needle tracking and insertion detection module measures the width and angles of the needle and the blood vessel and suggests if it is good to make a procedure or not by giving a visible marker.

According, to one embodiment herein, the processor includes a blood statistics module. In one embodiment the blood statistics module facilitates in recording the heart beat rate and the blood flow velocity of the subject under observation. In another embodiment the system (100) is provided with a distance variability and vein zooming module to enable a user for a detailed visualization of desired image.

According to one embodiment herein, a linear polarizer is used along with the wavelength filter (110) to generate a single plane at light. In an embodiment the light signal emitted from the light source (102) and the reflected light signal from the subject under observation (116) is passed through said linear polarized wavelength filter to allow at least one of X component and Y component of light. In one embodiment the linear polarizer (134) is a split polarizer. In one embodiment transmit and receive path polarizers could be arranged in a parallel form. In another embodiment transmit and receive path polarizers could be arranged in a cross form. In another embodiment the light source (102) could be a co-centric light source (102) consisting of multiple sources of light arranged in an array.

According to one embodiment herein, the light source (102) is made of a curved surface to facilitate clear and uniform illumination to the subject under observation. In one embodiment the curvature of the subject is determined based on multiple IR/UV/Proximity sensors placed on the system (100) and the measured curvature is used to adjust the curvature of the light source (102).

According to one embodiment herein, the processor (108) is configured to remove undesirable portions of subject under observation area from the generated image such as background of the image. In yet another embodiment, the processor (108) is programmed to facilitate post processing of the image in order to improve the image quality. In another embodiment, the processor (108) is configured to enable dynamic alignment of the display image with respect to the acquired image.

According to one embodiment herein, the system (100) could be integrated with the existing devices in order to facilitate comfortable usage. In another embodiment, the system (100) is provided in communication with the mobile phones that include but are not limited to smart-phone, Android based phones, iOS based phones and projector phones. Further, in another embodiment, the system (100) might be configured to utilize the features such as processor, display, projector and camera from the existing devices (mobile phones) to which the system (100) is coupled. In another embodiment, the system (100) is coupled or mounted on to the injection needle which is used for venipuncture. In another embodiment, the system (100) could be coupled with the devices such as goggles, head mount displays and heads-up displays.

According to one embodiment herein, the processor (108) is configured to display the generated image of the subject under observation (116) as a three dimensional image. The three dimensional image provides better visualization about the depth and width of the blood vessels. In an embodiment the blood vessels include arteries, veins and capillaries. In another embodiment, the depth and width of the vein could be identified by the two-dimensional images as well. Furthermore, the processor (108) is configured to indicate a point that is best suited for venipuncture in the generated image (vein map). In another embodiment, the point that is best suited for venipuncture is identified by vein width. In another embodiment, the point that is best suited for venipuncture is identified by at least one of vein depth, vein width, vein length, and straightness of the vein.

According to one embodiment herein, the processor (108) is adapted to facilitate analysis of blood and related fluids using the detailed blood specimen images of the identified blood vessel. The analysis of blood and related fluids may be enabled by the same image or different image which may be of different resolution. Further, the analysis results are displayed on the display device. Furthermore, the memory of processor (108) is configured to store all the information regarding the generated image, analysis results and so on which could be used for future studies. Further, the analyses include but are not limited to platelet count, red blood corpuscles count, sugar level analysis, glucose level analysis and so on.

It should be noted that the aforementioned configuration of system (100) is provided for the ease of understanding the embodiments herein. However, certain embodiments may have a different configuration of the components of the system (100) and certain other embodiments may exclude certain components of the system (100). For example the system (100) could be configured to generate video information of the subject under observation (116) instead of the image. Further, the processor (108) may include any other hardware device, combination of hardware devices, software devices or combination of hardware or software devices that could achieve one or more process discussed in the description. Therefore, such embodiments and any modification by addition or exclusion of certain components of the system (100) without otherwise deterring the intended function of the system (100) as is apparent from this description and drawings are also within the scope of this invention.

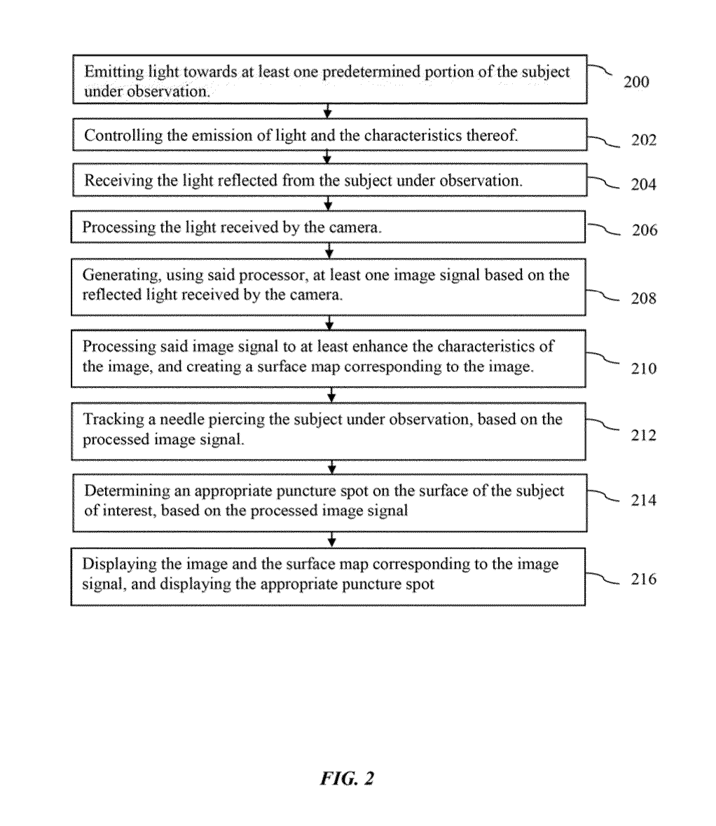

According to one embodiment herein, a method for locating and highlighting the blood vessels of a subject under observation and analyzing the blood characteristics thereof, has been explained herein below with reference to FIG. 2. The method includes the steps of emitting light towards at least one predetermined portion of the subject under observation, using a light source (step 200); controlling the emission of light and the characteristics thereof using a control unit (step 202); receiving the light reflected from the subject under observation, using a camera (step 204); processing the light received by the camera, using a processor (step 206); generating, using said processor, at least one image signal based on the reflected light received by the camera (step 208); processing said image signal to at least enhance the characteristics of the image, and creating a surface map corresponding to the image, using said processor (step 210); tracking a needle piercing the subject under observation, based on the processed image signal (step 212) determining an appropriate puncture spot on the surface of the subject of interest, based on the processed image signal (step 214); and displaying the image and the e surface map corresponding to the image signal, and displaying the appropriate puncture spot (step 216).

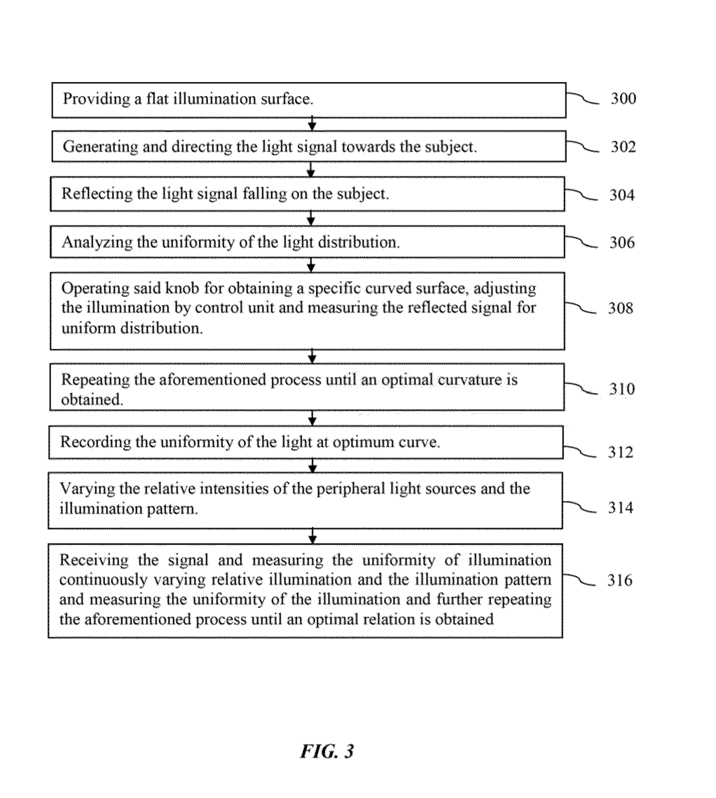

FIG. 3 is a flow chart depicting the steps involved in controlling the amount of light projected towards the subject under observation, according to one embodiment herein. The method includes the steps of: providing a flat illumination surface (step 301), generating and directing the light signal towards the subject (step 302), reflecting the light signal falling on the subject (step 303), analyzing the uniformity of the light distribution (step 304), operating said knob (step 305) for obtaining a specific curved surface, adjusting the illumination by control unit and measuring the reflected signal for uniform distribution (step 306), repeating the aforementioned process until an optimal curvature is obtained (step 307), recording the uniformity of the light at optimum curve step 308) varying the relative intensities of the peripheral light sources and the illumination pattern (step 309), receiving the signal and measuring the uniformity of illumination (step 310) continuously varying relative illumination and the illumination pattern and measuring the uniformity of the illumination and repeating the aforementioned process until an optimal relation is obtained (step 311).

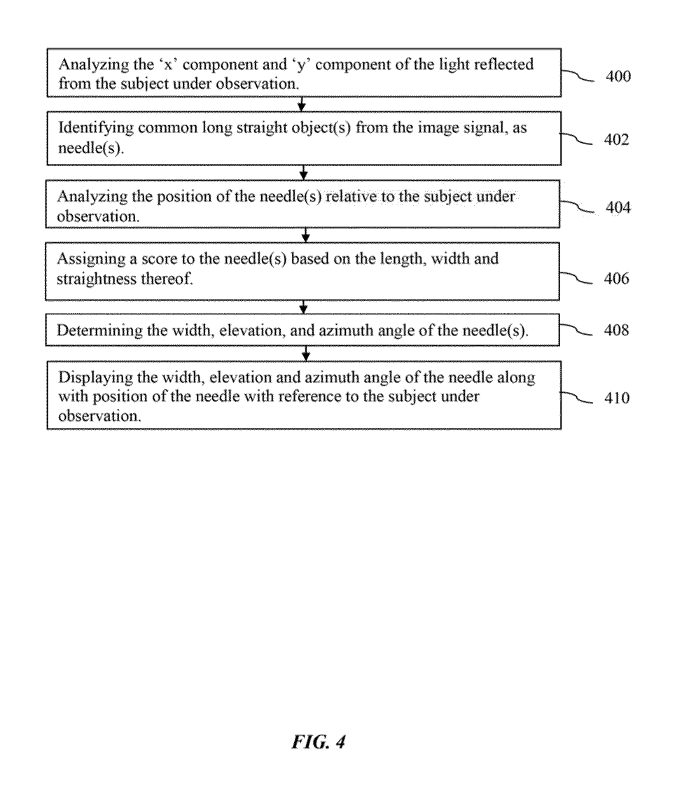

FIG. 4 is a flow chart illustrating the steps involved in tracking a needle piercing the subject under observation, according to one embodiment herein. Tracking a needle piercing the subject under observation includes the following steps: analyzing the ‘x’ component and ‘y’ component of the light reflected from the subject under observation (step 400); identifying common long straight object(s) from the image signal, as needle(s) (step 402); analyzing the position of the needle(s) relative to the subject under observation (step 404); assigning a score to the needle(s) based on the length, width and straightness thereof (step 406): determining the width, elevation, and azimuth angle of the needle(s) (step 408), and displaying the width, elevation and azimuth angle of the needle along with position of the needle with reference to the subject under observation (step 410).

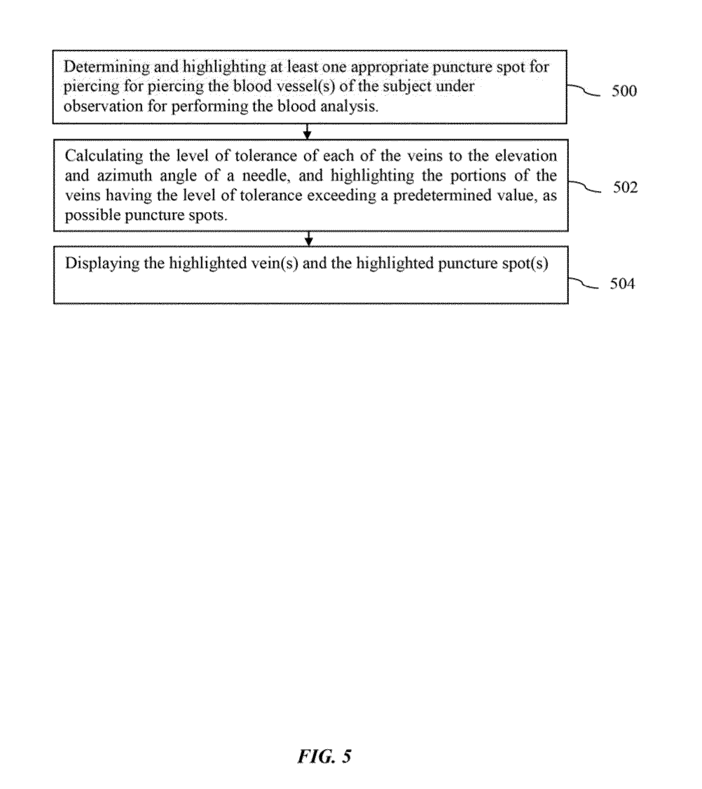

FIG. 5 depicts a flow chart illustrating the steps involved in statically determining an appropriate puncture spot on the surface of the subject of interest, according to one embodiment herein. The step of statically determining the puncture spot further includes the following steps: determining and highlighting at least one appropriate puncture spot for piercing for piercing the blood vessel(s) of the subject under observation for performing the blood analysis (step 500); calculating the level of tolerance of each of the veins to the elevation and azimuth angle of a needle, and highlighting the portions of the veins having the level of tolerance exceeding a predetermined value, as possible puncture spots (step 502); displaying the highlighted vein(s) and the highlighted puncture spot(s) (step 504).

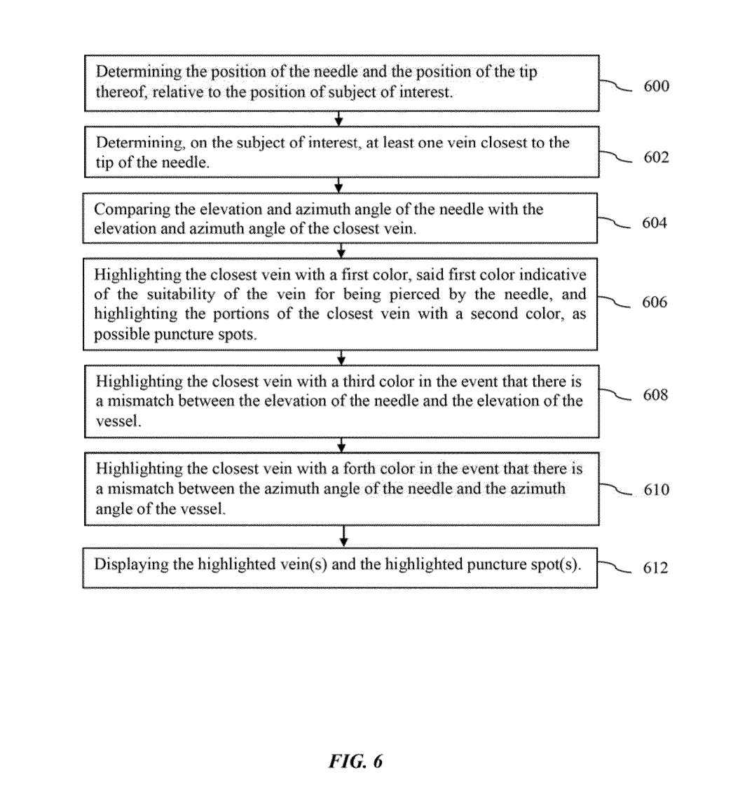

According to one embodiment herein, the step of determining an appropriate puncture spot on the surface of the subject of interest, further includes the step of dynamically determining an appropriate puncture spot. The step of dynamically determining an appropriate puncture spot further includes the following steps (as shown in FIG. 6): determining the position of the needle and the position of the tip thereof, relative to the position of subject of interest (step 600); determining, on the subject of interest, at least one vein closest to the tip of the needle (step 602); comparing the elevation and azimuth angle of the needle with the elevation and azimuth angle of the closest vein (step 604); highlighting the closest vein with a first color, said first color indicative of the suitability of the vein for being pierced by the needle, and highlighting the portions of the closest vein with a second color, as possible puncture spots (step 606); highlighting the closest vein with a third color in the event that there is a mismatch between the elevation of the needle and the elevation of the vessel (step 608); highlighting the closest vein with a forth color in the event that there is a mismatch between the azimuth angle of the needle and the azimuth angle of the vessel (step 610); and displaying the highlighted vein(s) and the highlighted puncture spot(s) (step 612).

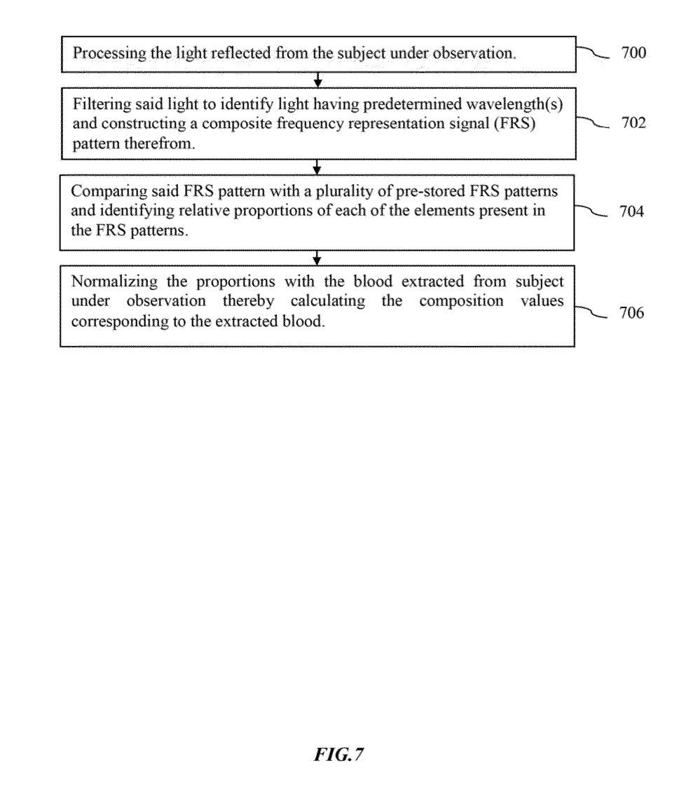

FIG. 7 depicts a flow chart illustrating the steps involved in analyzing the blood composition of the subject under observation, according to one embodiment herein. The analysis of blood composition, in accordance with the present disclosure includes the following steps: processing the light reflected from the subject under observation (steps 700); filtering said light to identify light having predetermined wavelength(s) and constructing a composite frequency representation signal (FRS) pattern therefrom (step 702); comparing said FRS pattern with a plurality of pre-stored FRS patterns and identifying relative proportions of each of the elements present in the FRS patterns (step 704); and normalizmg the proportions with the blood extracted from subject under observation thereby calculating the composition values corresponding to the extracted blood (step 706).

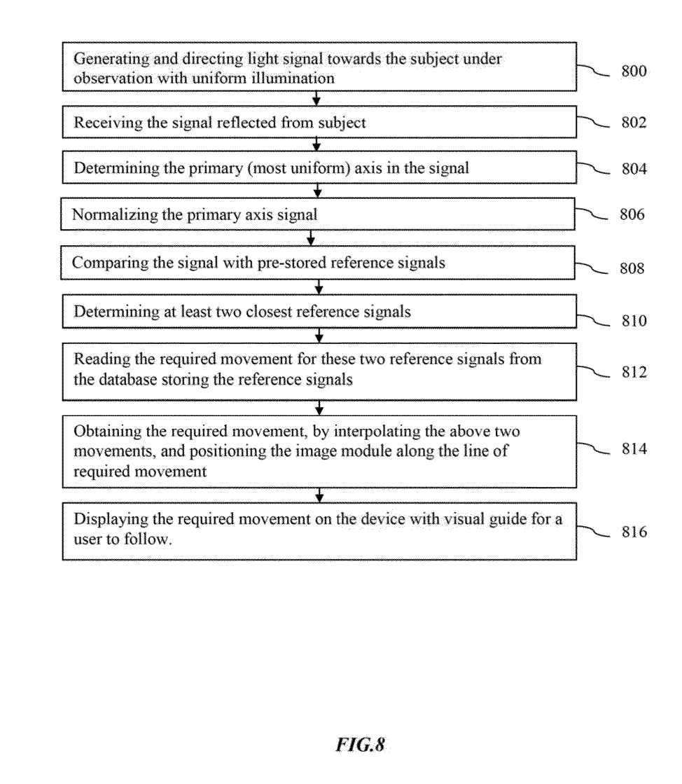

FIG. 8 depicts a flow chart illustrating the steps involved in automatic positioning of the imaging module for capturing an image having clarity, according to one embodiment herein. The automatic positioning of the imaging module includes the following steps: generating and directing light signal towards the subject under observation with uniform illumination (step 800), receiving the signal reflected from subject (step 802), determining the primary (most uniform) axis in the signal (step 804), normalizing the primary axis signal (step 806), comparing the signal with pre-stored reference signals (step 808), determining at least two closest reference signals (step 810), reading the required movement for these two reference signals from the database storing the reference signals (step 812), obtaining the required movement, by interpolating the above two movements, and positioning the image module along the line of required movement (step 814), and displaying the required movement on the device with visual guide for a user to follow (step 816).

It should be noted that the aforementioned steps have been provided for the ease of understanding of the embodiments of the invention. However, various steps provided in the above method may be performed in the order presented, in a different order, or simultaneously. Further, in some embodiments, one or more steps listed in the above method may be omitted. Therefore, such embodiments and any modification that is apparent from this description and drawings are also within the scope of this invention.

The foregoing description, of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A system for locating and highlighting the blood vessels of a subject under observation and analyzing the blood characteristics thereof, said system comprising:
an imaging module comprising:
at least one light source configured to emit light towards at least one predetermined portion of the subject under observation;
a control unit communicably coupled to said light source, said control unit configured to control the emission of light and the characteristics thereof; and
at least one camera configured to receive the light reflected from the subject under observation;
a processor cooperating with said imaging module, said processor configured to process the light received by the camera, said processor further configured to generate at least one image signal based on the reflected light received by the camera, said processor further configured to process said image signal to at least enhance the characteristics of the image, said processor still further configured to create a surface map corresponding to the image, said processor still further configured to track a needle piercing the subject under observation, based on the processed image signal, said processor still further configured to determine an appropriate puncture spot on the surface of the subject of interest, based on the processed image signal; and
a display module accessible to a user, said display module cooperating with said processor to receive said image signal and configured to display the image corresponding to the received image signal, said display module further configured to display the surface map corresponding to the received image signal.

2. The system as claimed in claim 1, wherein said control unit is further configured to control at least one of the intensity, pattern, curvature and wavelength of the light being emitted from the light source.

3. The system as claimed in claim 2, wherein said control unit is further configured to control at least one of the intensity, pattern, curvature and wavelength of the light reflected from the subject under observation, based on at least the skin tone, curvature and composition of the subject under observation.

4. The system as claimed in claim 1, wherein said system further comprises a diffuser filter and a polarizer filter, said diffuser filter being located in the path of the light emitted from the light source, said polarizer filter being located in the path of the light being reflected from the subject under observation.

5. The system as claimed in claim 1, wherein said processor cooperates with the display module to facilitate frame segmentation of the image generated from the image signal, said processor further configured to identify and highlight the regions of interest in the generated image.

6. The system as claimed in claim 1, wherein the processor is further configured to improve the signal-to-noise ratio (SNR) of the image signal, said processor still further configured to selectively modify the contrast of the image to increase the visibility of the regions of interest.

7. The system as claimed in claim 1, wherein said system further includes a Previous Frame Feedback Module (PFF module), said PFF module configured to store the information corresponding to the characteristics of previously generated images, said PFF module further configured to analyze the stored information, and use the analyzed information to selectively enhance the characteristics of a currently generated image signal.

8. The system as claimed in claim 1, wherein said processor is further configured to track and detect needle, piercing the subject under observation, said processor cooperating with the camera to analyze the ‘x’ component and ‘y’ component of light reflected from the subject under observation, said processor still further configured to determine common long straight object(s) from the image signal, as needle(s), said processor still further configured to analyze the position of the needle(s) relative to the subject under observation, said processor still further configured to assign a score to the needle(s) based on the length, shape, width and straightness thereof, said processor further configured to determine the width, elevation, and azimuth angle of the needle(s), said processor further configured to cooperate with the display module to display the width, elevation and azimuth angle of the needle along with position of the needle with reference to the subject under observation.

9. The system as claimed in claim 1, wherein said processor is further configured to statically determine and highlight at least one appropriate puncture spot for piercing the blood vessel(s) of the subject under observation for performing at least one of blood analysis, fluid injection and blood draw, said processor configured to calculate the level of tolerance of each of the veins to the elevation and azimuth angle of a needle, said processor still, further configured to highlight the portions of the veins having the level of tolerance exceeding a predetermined value, as possible puncture spots, said processor further configured to cooperate with the display module to display the highlighted vein(s) and the highlighted puncture spot(s).

10. The system as claimed in claim 9, wherein said processor is further configured to dynamically determine and highlight at least one appropriate puncture spot for piercing the blood vessels of the subject under observation, said processor still further configured to:
determine the position of the needle and the position of the tip thereof, relative to the position of subject of interest;
determine, on the subject of interest, at least one vein closest to the tip of the needle;
compare the elevation and azimuth angle of the needle with the elevation and azimuth angle of the closest vein;
highlight the closest vein with a first color, said first color indicative of the suitability of the vein for being pierced by the needle, and highlight the portions of the closest vein with a second color, as possible puncture spots;
highlight the closest vein with a third color in the event that there is a mismatch between the elevation of the needle and the elevation of the vessel; and
highlight the closest vein with a forth color in the event that there is a mismatch between the azimuth angle of the needle and the azimuth angle of the vessel;
said processor further configured to cooperate with the display module to display the highlighted vein(s) and the highlighted puncture spot(s).

11. The system as claimed in claim 1, wherein said processor is further configured to analyze the blood extracted from the subject under observation, said processor cooperating with the camera to access and process the light reflected from the subject under observation, said processor still further configured to filter said light to identify light having predetermined, wavelength(s) and construct a composite frequency representation signal (FRS) pattern therefrom, said processor still further configured to compare said FRS pattern with a plurality of pre-stored FRS patterns and identify relative proportions of each of the elements present in the FRS patterns, said processor still further configured to normalize the proportions with the blood extracted from subject under observation thereby calculating the composition values corresponding to the extracted blood.

12. A method for locating and highlighting the blood vessels of a subject under observation and analyzing the blood characteristics thereof, said method comprising the following steps:
emitting light towards at least one predetermined portion of the subject under observation, using a light source;
controlling the emission of light and the characteristics thereof, using a control unit:
receiving the light reflected from the subject under observation, using a camera;
processing the light received by the camera, using a processor;
generating, using said processor, at least one image signal based on the reflected light received by the camera;
processing said image signal to at least enhance the characteristics of the image, and creating a surface map corresponding to the image, using said processor;
tracking a needle piercing the subject under observation, based on the processed image signal;
determining an appropriate puncture spot on the surface of the subject of interest, based on the processed image signal; and
displaying the image and the surface map corresponding to the image signal, and displaying the appropriate puncture spot.

13. The method as claimed in claim 12, wherein the step of controlling the emission of light and the characteristics thereof, further includes the step of controlling at least one of the intensity, pattern, curvature and wavelength of the light, based on skin tone, curvature and composition of the subject under observation.

14. The method as claimed in claim 12, wherein the method further includes the step of facilitating frame segmentation of the image generated from the image signal, and identifying and highlighting the regions of interest in the generated image.

15. The method as claimed in claim 12, wherein the method further includes the steps of improving, the signal-to-noise ratio (SNR) of the image signal using the processor, and selectively modify the contrast of the image to increase the visibility of the regions of interest, using the processor.

16. The method as claimed in claim 12, wherein the method further includes the steps of storing the information corresponding to the characteristics of previously generated images, analyzing the stored information, and using the analyzed information to selectively enhance the characteristics of a currently generated image signal.

17. The method as claimed in claim 12, wherein the step of tracking a needle piercing the subject under observation, further includes the following steps:
analyzing the ‘x’ component and ‘y’ component of the light reflected from the subject wider observation;
identifying common long straight object(s) from the image signal, as needle(s);
analyzing the position of the needle(s) relative to the subject under observation;
assigning a score to the needle(s) based on the length, width and straightness thereof;
determining the width, elevation, and azimuth angle of the needle(s); and
displaying the width, elevation and azimuth angle of the needle along with position of the needle with reference to the subject under observation.

18. The method as claimed in claim 12, wherein the step of determining an appropriate puncture spot on the surface of the subject of interest, further includes the step of statically determining an appropriate puncture spot, said step further comprising the following steps:
determining and highlighting at least one appropriate puncture spot for piercing for piercing the blood vessel(s) of the subject under observation for performing the blood analysis;
calculating the level of tolerance of each of the veins to the elevation and azimuth angle of a needle, and highlighting the portions of the veins having the level of tolerance exceeding: a predetermined value, as possible puncture spots; and
displaying the highlighted vein(s) and the highlighted puncture spot(s).

19. The method as claimed in claim 12, wherein the step of determining an appropriate puncture spot on the surface of the subject of interest, further includes the step of dynamically determining an appropriate puncture spot, said step further comprising the following steps:
determining the position of the needle and the position of the tip thereof, relative to the position of subject of interest;
determining, on the subject of interest, at least one vein closest to the tip of the needle;
comparing the elevation and azimuth angle of the needle with the elevation and azimuth angle of the closest vein;
highlighting the closest vein with a first color, said first color indicative of the suitability of the vein for being pierced by the needle, and highlighting the portions of the closest vein with a second color, as possible puncture spots;
highlighting the closest vein with a third color in the event that there is a mismatch between the elevation of the needle and the elevation of the vessel;
highlighting the closest vein with a forth color in the event that there is a mismatch between the azimuth angle of the needle and the azimuth angle of the vessel; and
displaying the highlighted vein(s) and the highlighted puncture spot(s).

20. The method as claimed in claim 12, wherein the method further includes the step of analyzing the blood extracted from the subject under observation, said step further comprising the following steps:
processing the light reflected from the subject under observation;
filtering said light to identify light having predetermined wavelength(s) and constructing a composite frequency representation signal (FRS) pattern therefrom;
comparing said FRS pattern with a plurality of pre-stored FRS patterns and identifying relative proportions of each of the elements present in the FRS patterns; and
normalizing the proportions with the blood extracted from subject under observation thereby calculating the composition values corresponding to the extracted blood.
